(19) United States
(12) Patent Application Publication
(10) Pub. No.: US 2019/0270956 A1
(43) Pub. Date: Sep. 5, 2019

(54) MULTIPLE FLOW PATH CELL CULTIVATION METHOD

(71) Applicant: UBE INDUSTRIES, LTD., Yamaguchi (JP)

(72) Inventors: Masahiko HAGIHARA, Yamaguchi (JP); Shinsaku FUSE, Yamaguchi (JP); Motohisa SHIMIZU, Yamaguchi (JP); Yukinori WADA, Yamaguchi (JP)

(21) Appl. No.: 16/320,413

(22) PCT Filed: Jul. 25, 2017
(86) PCT No.: PCT/JP2017/026944

§ 371 Date: Jan. 24, 2019

(30) Foreign Application Priority Data
Jul. 25, 2016 (JP) 2016-145776

(51) Int. Cl. C12M 1/00; C12N 5/00

(52) U.S. Cl. CPC C12M 23/20; C12M 23/02; C12M 23/34; C12N 5/0068; C12M 29/00

(57) ABSTRACT

Provided are a cell cultivation device comprising a polymer porous film, and a cell cultivation method using said cell cultivation device. The cell cultivation device comprises: two or more levels of parallel or antiparallel flat flow paths; a polymer porous film that is positioned in the flat flow paths; a culture medium supply means that is positioned at one end of the flat flow paths; a culture medium discharge means that is positioned at the other end of the flat flow paths; a head tank that is positioned at an upper section of the flat flow path of the uppermost level; a culture medium distribution means that distributes a culture broth from the head tank to the flat flow paths; and a culture medium recovery means that recovers the culture broth that is discharged from the culture medium discharge means.

### FIELD

The present invention relates to a cell culture device comprising a porous polymer film, and further relates to a cell culture method using the cell culture device comprising the porous polymer film.

### BACKGROUND

In recent years, proteins such as enzymes, hormones, antibodies, cytokines, viruses (viral proteins) used for treatment and vaccine are industrially produced using cultured cells. However, such a protein production technology is expensive, raising medical cost. Accordingly, there have been demands for innovating technologies for culturing cells at high density and for increasing protein production, aiming at great reduction of cost.

As cells for protein production, anchorage-dependent adherent cells which adhere to a culture substrate may be sometimes used. Since such cells grow anchorage-dependently, they need to be cultured while being adhered onto the surface of a dish, plate or chamber. Conventionally, in order to culture such adherent cells in a large amount, it was preferable to increase the surface area to be adhered. However, increasing the culturing area inevitably requires to increase the space, which is responsible for increase in cost.

As a method to culture a large amount of adherent cells while decreasing the culture space, a method for culture using a microporous carrier, especially a microcarrier, has been developed (for example, PTL 1). In a cell culturing system using microcarriers, it is preferable to carry out sufficient stirring and diffusion so that the microcarriers do not aggregate together. Since this requires a volume allowing adequate agitation and diffusion of the medium in which the microcarriers are dispersed, there is an upper limit to the density at which the cells can be cultured. In order to separate the microcarrier from the medium, separation is preferably performed using a filter which can separate fine particles, possibly resulting in increased cost. Considering the foregoing, there is a demand for innovative methodology for cell culture which cultures cells at high density.

<Porous Polyimide Film>

Porous polyimide films have been utilized in the prior art for filters and low permittivity films, and especially for battery-related purposes, such as fuel cell electrolyte membrane and the like. PTLs 2 to 4 describe porous polyimide films with numerous macrovoids, having excellent permeability to objects such as gases, high porosity, excellent smoothness on both surfaces, relatively high strength and, despite high porosity, excellent resistance against compression stress in the film thickness direction. All of these are porous polyimide films formed via amic acid.

The cell culture method which includes applying cells to a porous polyimide film and culturing them is reported (PTL 5).

### CITATION LIST

### Patent Literature

[PTL 1] WO2003/054174

[PTL 2] WO2010/038873

[PTL 3] Japanese Unexamined Patent Publication (Kokai) No. 2011-219585

[PTL 4] Japanese Unexamined Patent Publication (Kokai) No. 2011-219586

[PTL 5] WO2015/012415

### SUMMARY

### Technical Problem

The object of the present invention is to provide a cell culture device comprising a porous polymer film and to provide a cell culture method using the cell culture device comprising the porous polymer film.

### Solution to Problem

The inventors of the present invention discovered that a porous polymer film having a predetermined structure not only provides an optimal space in which a large number of cells can be cultured, but also provides a moist environment that is resistant to drying and completed a device that cultures cells by the exposure thereof to a gas phase and a culture method using same. In other words, the present invention preferably includes, but is not limited to, the following modes.

[1] A cell culture device characterized by comprising:
two or more-step parallel planar flow paths;
a porous polymer film disposed in the planar flow paths;
a medium supplying means disposed at one end of each of the planar flow paths;
a medium discharging means disposed at the other end of each of the planar flow paths;
a head tank disposed at the top of the uppermost planar flow path;
a medium distributing means for distributing the culture liquid from the head tank to each planar flow path; and
a medium collecting means for collecting the culture liquid discharged from the medium discharging means;

wherein the porous polymer films are a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B;

wherein the macrovoid layer has a partition wall bonded to the surface layers A and B, and a plurality of macrovoids surrounded by such a partition wall and the surface layers A and B; and

wherein the culture medium continuously flows from the medium supplying means to the medium discharging means via the planar flow path.

[2] A cell culture device characterized by comprising:
two or more-step antiparallel planar flow paths;
a porous polymer film disposed in the planar flow paths;
a medium supplying means disposed at one end of each of the planar flow paths;
a medium discharging means disposed at the other end of each of the planar flow paths;
a head tank disposed at the top of the uppermost planar flow path;
a medium distributing means for distributing the culture liquid from the head tank to each of uppermost planar flow path; and
a medium collecting means for collecting the culture liquid discharged from the lowermost;

wherein the porous polymer film is a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B;

wherein the macrovoid layer has a partition wall bonded to the surface layers A and B, and a plurality of macrovoids surrounded by such a partition wall and the surface layers A and B; and

wherein the culture liquid continuously flows from the medium supplying means to the medium discharging means via the planar flow path, and furthermore the culture liquid discharged from the medium discharging means is poured into the medium supplying means installed in the next stage planar flow path.

[3] The cell culture device according to [1] or [2], wherein the planar flow path is inclined at 0 to 20° from a horizontal plane.
[4] The cell culture device according to any one of [1] to [3], the device further comprising:
a medium discharging line communicating with an outlet of the medium collecting means; and
a medium supplying line communicating with a supply port disposed at an upper portion of the head tank;

wherein the other end of the medium discharging line is communicated with the other end of the medium supplying line via a pump, and the culture liquid is circulatable.

[5] The cell culture device according to any one of [1] to [4], wherein the medium distributing means is a mist supplying nozzle.
[6] The cell culture device according to any one of [1] to [4], wherein the planar flow paths are each installed within the thin bag.
[7] The cell culture device according to any one of [1] to [6], wherein the porous polymer films are installed in the plane flow path, with the films:

i) being folded up;

ii) being wound into a roll-like shape;

iii) concatenating sheets or pieces thereof with a thread-like structure;

iv) being tied together into a rope-like shape; and/or

v) two or more thereof being stacked.

[8] The cell culture device according to any one of [1] to [7], wherein the porous polymer films are modularized porous polymer films fitted with a casing;

wherein the modularized porous polymer films fitted with a casing are contained within the casing with:

(i) the two or more independent porous polymer films being aggregated;

(ii) the porous polymer films being folded up;

(iii) the porous polymer films being wound into a roll-like shape; and/or

(iv) the porous polymer films being tied together into a rope-like shape;

wherein the modularized porous polymer films fitted with the casing are installed in the planar flow path.

[9] The cell culture device according to any one of [1] to [8], wherein the porous polymer film has a plurality of pores having an average pore diameter of 0.01 to 100 μm.
[10] The cell culture device according to any one of [1] to [9], wherein an average pore diameter of the surface layer A is 0.01 to 50 μm.
[11] The cell culture device according to any one of [1] to [10], wherein an average pore diameter of the surface layer B is 20 to 100 μm.
[12] The cell culture device according to any one of [1] to [11], wherein a total film thickness of the porous polymer film is 5 to 500 μm.
[13] The cell culture device according to any one of [1] to [12], wherein the porous polymer film is a porous polyimide film.
[14] The method according to [13], wherein the porous polyimide film is a porous polyimide film comprising a polyimide derived from tetracarboxylic dianhydride and diamine.
[15] The cell culture device according to [13] or [14], wherein the porous polyimide film is a colored porous polyimide film that is obtained by molding a polyamic acid solution composition comprising a polyamic acid solution derived from tetracarboxylic dianhydride and diamine, and a coloring precursor, and subsequently heat-treating the resultant composition at 250° C. or higher.
[16] The cell culture device according to any one of [1] to [12], wherein the porous polymer film is a porous polyethersulfone film.
[17] A method for culturing a cell which uses the cell culture device according to any one of [1] to [16].

### Advantageous Effects of Invention

The present invention allows simple and efficient continuous cell culturing even under conditions requiring little space and little culture medium by using a porous polymer film as a cell culture support. Further, as the porous polymer film has pores with slightly hydrophilic properties, stable liquid retention is achieved within the porous polymer film and a moist environment that is resistant to drying is maintained. Thus, survival and proliferation of cells can be achieved even with very little medium compared to conventional cell culture devices. Furthermore, as culturing is possible even if a part of or the entire porous polymer film is exposed to air, oxygen can be efficiently supplied to the cells and a large number of cells can be cultured.

According to the present invention, a large number of cells can be stably cultured over a long period in a small volume using a system which continuously supplies culture liquid through planar flow paths (inclined 0 to 10° to the horizontal plane), on which porous polymer films are provided, that are repeated parallelly or antiparallelly and over which culture liquid is flowed so as to be recirculated. Further, as a liquid reservoir (head tank) that store culture liquid is separated from the cells, when, for example, adjustments such as adjustments in pH or in the addition of glucose are made, even vigorous stirring, foaming and the like, which are disapproved of with respect to cells, can be performed, and thus is highly advantageous in view of chemical engineering.

### BRIEF DESCRIPTION OF DRAWINGS

FIG. 1 is a diagram illustrating a parallel-type cell culture device according to an embodiment.

FIG. 2 is a diagram illustrating an embodiment of a parallel-type cell culture device.

FIG. 3(A) is a diagram illustrating an embodiment of a cascade-type cell culture device.

FIG. 4 is a drawing of a cell culture model using a porous polymer.

### DETAILED DESCRIPTION OF INVENTION

The embodiments of the present invention will be described below referring to the drawings as needed. The configurations of these embodiments are exemplary, and the configurations of the present invention are not limited thereto.

### 1. Porous Polymer Film

An average pore diameter of the pore present on a surface layer A (hereinafter referred to as “surface A” or “mesh surface”) in the porous polymer film used for the present invention is not particularly limited, but is, for example, 0.01 μm or more and less than 200 μm, 0.01 to 150 μm, 0.01 to 100 μm, 0.01 to 50 μm, 0.01 to 40 μm, 0.01 to 30 μm, 0.01 to 20 μm, or 0.01 to 15 μm, preferably 0.01 to 15 μm.

The average pore diameter of the pore present on a surface layer B (hereinafter referred to as “surface B” or “large pore surface”) in the porous polymer film used for the present invention is not particularly limited so long as it is larger than the average pore diameter of the pore present on the surface A, but is, for example, greater than 5 μm and 200 μm or less, 20 μm to 100 μm, 30 μm to 100 μm, 40 μm to 100 μm, 50 μm to 100 μm, or 60 μm to 100 μm, preferably 20 μm to 100 μm.

The average pore diameter on the surface of the porous polymer film is determined by measuring pore area for 200 or more open pore portions, and calculated an average diameter according to the following Equation (1) from the average pore area assuming the pore shape as a perfect circle.

[Math. 1]

Average Pore Diameter=2×\sqrt{(Sa/π)} (1)

(wherein Sa represents the average value for the pore areas)

The thicknesses of the surface layers A and B are not particularly limited, but is, for example, 0.01 to 50 μm, preferably 0.01 to 20 μm.

The average pore diameter of macrovoids in the planar direction of the film in the macrovoid layer in the porous polymer film is not particularly limited but is, for example, 10 to 500 μm, preferably 10 to 100 μm, and more preferably 10 to 80 μm. The thicknesses of the partition wall in the macrovoid layer are not particularly limited, but is, for example, 0.01 to 50 μm, preferably 0.01 to 20 μm. In an embodiment, at least one partition wall in the macrovoid layer has one or two or more pores connecting the neighboring macrovoids and having the average pore diameter of 0.01 to 100 μm, preferably 0.01 to 50 μm. In another embodiment, the partition wall in the macrovoid layer has no pore.

The total film thickness of the porous polymer film used for the invention is not particularly limited, but may be 5 μm or more, 10 μm or more, 20 μm or more or 25 μm or more, and 500 μm or less, 300 μm or less, 100 μm or less, 75 μm or less, or 50 μm or less. It is preferably 5 to 500 μm, and more preferably 25 to 75 μm.

The film thickness of the porous polymer film used for the invention can be measured using a contact thickness gauge.

The porosity of the porous polymer film used in the present invention is not particularly limited but is, for example, 40% or more and less than 95%.

The porosity of the porous polymer film used for the invention can be determined by measuring the film thickness and mass of the porous film cut out to a prescribed size, and performing calculation from the basis weight according to the following Equation (2).

[Math. 2]

Porosity (%)=(1−w/(S×d×D)×100 (2)

(wherein S represents the area of the porous film, d represents the total film thickness, w represents the measured mass, and D represents the polymer density. The density is defined as 1.34 g/cm³ when the polymer is a polyimide.)

The porous polymer film used for the present invention is preferably a porous polymer film which includes a three-layer structure porous polymer film having a surface layer A and a surface layer B, the surface layers having a plurality of pores, and a macrovoid layer sandwiched between the surface layers A and B; wherein the average pore diameter of the pore present on the surface layer A is 0.01 μm to 15 μm, and the average pore diameter of the pore present on the surface layer B is 20 μm to 100 μm; wherein the macrovoid layer has a partition wall bonded to the surface layers A and B, and a plurality of macrovoids surrounded by such a partition wall and the surface layers A and B, the thickness of the macrovoid layer, and the surface layers A and B is 0.01 to 20 μm; wherein the pores on the surface layers A and B communicate with the macrovoid, the total film thickness is 5 to 500 μm, and the porosity is 40% or more and less than 95%. In an embodiment, at least one partition wall in the macrovoide layer has one or two or more pores connecting the neighboring macrovoids with each other and having the average pore diameter of 0.01 to 100 μm, preferably 0.01 to 50 μm. In another embodiment, the partition wall does not have such pores.

The porous polymer film used for the present invention is preferably sterilized. The sterilization treatment is not particularly limited, but any sterilization treatment such as dry heat sterilization, steam sterilization, sterilization with a disinfectant such as ethanol, electromagnetic wave sterilization such as ultraviolet rays or gamma rays, and the like can be mentioned.

The porous polymer film used for the present invention is not particularly limited so long as it has the structural features described above and includes, preferably a porous polyimide film or porous polyethersulfone film.

1-1. Porous Polyimide Film

Polyimide is a general term for polymers containing imide bonds in the repeating unit, and usually it refers to an aromatic polyimide in which aromatic compounds are directly linked by imide bonds. An aromatic polyimide has an aromatic-aromatic conjugated structure via an imide bond, and therefore has a strong rigid molecular structure, and since the imide bonds provide powerful intermolecular force, it has very high levels of thermal, mechanical and chemical properties.

The porous polyimide film usable for the present invention is a porous polyimide film preferably containing polyimide (as a main component) obtained from tetracarboxylic dianhydride and diamine, more preferably a porous polyimide film composed of tetracarboxylic dianhydride and diamine. The phrase “including as the main component” means that it essentially contains no components other than the polyimide obtained from a tetracarboxylic dianhydride and a diamine, as constituent components of the porous polyimide film, or that it may contain them but they are additional components that do not affect the properties of the polyimide obtained from the tetracarboxylic dianhydride and diamine.

In an embodiment, the porous polyimide film usable for the present invention includes a colored porous polyimide film obtained by forming a polyamic acid solution composition including a polyamic acid solution obtained from a tetracarboxylic acid component and a diamine component, and a coloring precursor, and then heat treating it at 250° C. or higher.

A polyamic acid is obtained by polymerization of a tetracarboxylic acid component and a diamine component. A polyamic acid is a polyimide precursor that can be cyclized to a polyimide by thermal imidization or chemical imidization.

The polyamic acid used may be any one that does not have an effect on the invention, even if a portion of the amic acid is imidized. Specifically, the polyamic acid may be partially thermally imidized or chemically imidized.

When the polyamic acid is to be thermally imidized, there may be added to the polyamic acid solution, if necessary, an imidization catalyst, an organic phosphorus-containing compound, or fine particles such as inorganic fine particles or organic fine particles. In addition, when the polyamic acid is to be chemically imidized, there may be added to the polyamic acid solution, if necessary, a chemical imidization agent, a dehydrating agent, or fine particles such as inorganic fine particles or organic fine particles. Even if such components are added to the polyamic acid solution, they are preferably added under conditions that do not cause precipitation of the coloring precursor.

In this specification, a “coloring precursor” is a precursor that generates a colored substance by partial or total carbonization under heat treatment at 250° C. or higher.

Coloring precursors usable for the production of the porous polyimide film are preferably uniformly dissolved or dispersed in a polyamic acid solution or polyimide solution and subjected to thermal decomposition by heat treatment at 250° C. or higher, preferably 260° C. or higher, even more preferably 280° C. or higher and more preferably 300° C. or higher, and preferably heat treatment in the presence of oxygen such as air, at 250° C., preferably 260° C. or higher, even more preferably 280° C. or higher and more preferably 300° C. or higher, for carbonization to produce a colored substance, more preferably producing a black colored substance, with carbon-based coloring precursors being most preferred.

The coloring precursor, when being heated, first appears as a carbonized compound, but compositionally it contains other elements in addition to carbon, and also includes layered structures, aromatic crosslinked structures and tetrahedron carbon-containing disordered structures.

Carbon-based coloring precursors are not particularly restricted, and for example, they include tar or pitch such as petroleum tar, petroleum pitch, coal tar and coal pitch, coke, polymers obtained from acrylonitrile-containing monomers, ferrocene compounds (ferrocene and ferrocene derivatives), and the like. Of these, polymers obtained from acrylonitrile-containing monomers and/or ferrocene compounds are preferred, with polyacrylonitrile being preferred as a polymer obtained from an acrylonitrile-containing monomer.

Moreover, in another embodiment, examples of the porous polyimide film which may be used for the preset invention also include a porous polyimide film which can be obtained by molding a polyamic acid solution derived from a tetracarboxylic acid component and a diamine component followed by heat treatment without using the coloring precursor.

The porous polyimide film produced without using the coloring precursor may be produced, for example, by casting a polyamic acid solution into a film, the polyamic acid solution being composed of 3 to 60% by mass of polyamic acid having an intrinsic viscosity number of 1.0 to 3.0 and 40 to 97% by mass of an organic polar solvent, immersing or contacting in a coagulating solvent containing water as an essential component, and imidating the porous film of the polyamic acid by heat treatment. In this method, the coagulating solvent containing water as an essential component may be water, or a mixed solution containing 5% by mass or more and less than 100% by mass of water and more than 0% by mass and 95% by mass or less of an organic polar solvent. Further, after the imidation, one surface of the resulting porous polyimide film may be subjected to plasma treatment.

The tetracarboxylic dianhydride which may be used for the production of the porous polyimide film may be any tetracarboxylic dianhydride, selected as appropriate according to the properties desired. Specific examples of tetracarboxylic dianhydrides include biphenyltetracarboxylic dianhydrides such as pyromellitic dianhydride, 3,3′,4,4′-biphenyltetracarboxylic dianhydride (s-BPDA) and 2,3,3′,4′-biphenyltetracarboxylic dianhydride (a-BPDA), oxydiphthalic dianhydride, diphenylsulfone-3,4,3′,4′-tetracarboxylic dianhydride, bis(3,4-dicarboxyphenyl)sulfide dianhydride, 2,2-bis(3,4-dicarboxyphenyl)-1,1,1,3,3,3-hexafluoropropane dianhydride, 2,3,3′,4′-benzophenonetetracarboxylic dianhydride, 3,3′,4,4′-benzophenonetetracarboxylic dianhydride, bis(3,4-dicarboxyphenyl)methane dianhydride, 2,2-bis(3,4-dicarboxyphenyl)propane dianhydride, p-phenylenebis(trimellitic acid monoester acid anhydride), p-biphenylenebis(trimellitic acid monoester acid anhydride), m-terphenyl-3,4,3′,4′-tetracarboxylic dianhydride, p-terphenyl-3,4,3′,4′-tetracarboxylic dianhydride, 1,3-bis(3,4-dicarboxyphenoxy)benzene dianhydride, 1,4-bis(3,4-dicarboxyphenoxy)benzene dianhydride, 1,4-bis(3,4-dicarboxyphenoxy)biphenyl dianhydride, 2,2-bis[(3,4-dicarboxyphenoxy)phenyl]propane dianhydride, 2,3,6,7-naphthalenetetracarboxylic dianhydride, 1,4,5,8-naphthalenetetracarboxylic dianhydride, 4,4′-(2,2-hexafluoroisopropylidene)diphthalic dianhydride, and the like. Also preferably used is an aromatic tetracarboxylic acid such as 2,3,3′,4′-diphenylsulfonetetracarboxylic acid. These may be used alone or in appropriate combinations of two or more.

Particularly preferred among these are at least one type of aromatic tetracarboxylic dianhydride selected from the group consisting of biphenyltetracarboxylic dianhydride and pyromellitic dianhydride. As a biphenyltetracarboxylic dianhydride there may be suitably used 3,3′,4,4′-biphenyltetracarboxylic dianhydride.

As diamine which may be used for the production of the porous polyimide film, any diamine may be used. Specific examples of diamines include the following.

1) Benzenediamines with one benzene nucleus, such as 1,4-diaminobenzene(paraphenylenediamine), 1,3-diaminobenzene, 2,4-diaminotoluene and 2,6-diaminotoluene;

2) diamines with two benzene nuclei, including diaminodiphenyl ethers such as 4,4′-diaminodiphenyl ether and 3,4′-diaminodiphenyl ether, and 4,4′-diaminodiphenylmethane, 3,3′-dimethyl-4,4′-diaminobiphenyl, 2,2′-dimethyl-4,4′-diaminobiphenyl, 2,2′-bis(trifluoromethyl)-4,4′-diaminobiphenyl, 3,3′-dimethyl-4,4′-diaminodiphenylmethane, 3,3′-dicarboxy-4,4′-diaminodiphenylmethane, 3,3′,5,5′-tetramethyl-4,4′-diaminodiphenylmethane, bis(4-aminophenyl)sulfide, 4,4′-diaminobenzanilide, 3,3′-dichlorobenzidine, 3,3′-dimethylbenzidine, 2,2′-dimethylbenzidine, 3,3′-dimethoxybenzidine, 2,2′-dimethoxybenzidine, 3,3′-diaminodiphenyl ether, 3,4′-diaminodiphenyl ether, 4,4′-diaminodiphenyl ether, 3,3′-diaminodiphenyl sulfide, 3,4′-diaminodiphenyl sulfide, 4,4′-diaminodiphenyl sulfide, 3,3′-diaminodiphenylsulfone, 3,4′-diaminodiphenylsulfone, 4,4′-diaminodiphenylsulfone, 3,3′-diaminobenzophenone, 3,3′-diamino-4,4′-dichlorobenzophenone, 3,3′-diamino-4,4′-dimethoxybenzophenone, 3,3′-diaminodiphenylmethane, 3,4′-diaminodiphenylmethane, 4,4′-diaminodiphenylmethane, 2,2-bis(3-aminophenyl)propane, 2,2-bis(4-aminophenyl)propane, 2,2-bis(3-aminophenyl)-1,1,1,3,3,3-hexafluoropropane, 2,2-bis(4-aminophenyl)-1,1,1,3,3,3-hexafluoropropane, 3,3′-diaminodiphenyl sulfoxide, 3,4′-diaminodiphenyl sulfoxide and 4,4′-diaminodiphenyl sulfoxide;

3) diamines with three benzene nuclei, including 1,3-bis(3-aminophenyl)benzene, 1,3-bis(4-aminophenyl)benzene, 1,4-bis(3-aminophenyl)benzene, 1,4-bis(4-aminophenyl)benzene, 1,3-bis(4-aminophenoxy)benzene, 1,4-bis(3-aminophenoxy)benzene, 1,4-bis(4-aminophenoxy)benzene, 1,3-bis(3-aminophenoxy)-4-trifluoromethylbenzene, 3,3′-diamino-4-(4-phenyl)phenoxybenzophenone, 3,3′-diamino-4,4′-di(4-phenylphenoxy)benzophenone, 1,3-bis(3-aminophenyl sulfide)benzene, 1,3-bis(4-aminophenyl sulfide)benzene, 1,4-bis(4-aminophenyl sulfide)benzene, 1,3-bis(3-aminophenylsulfone)benzene, 1,3-bis(4-aminophenylsulfone)benzene, 1,4-bis(4-aminophenylsulfone)benzene, 1,3-bis[2-(4-aminophenyl)isopropyl]benzene, 1,4-bis[2-(3-aminophenyl)isopropyl]benzene and 1,4-bis[2-(4-aminophenyl)isopropyl]benzene;

4) diamines with four benzene nuclei, including 3,3′-bis(3-aminophenoxy)biphenyl, 3,3′-bis(4-aminophenoxy)biphenyl, 4,4′-bis(3-aminophenoxy)biphenyl, 4,4′-bis(4-aminophenoxy)biphenyl, bis[3-(3-aminophenoxy)phenyl]ether, bis[3-(4-aminophenoxy)phenyl]ether, bis[4-(3-aminophenoxy)phenyl]ether, bis[4-(4-aminophenoxy)phenyl]ether, bis[3-(3-aminophenoxy)phenyl]ketone, bis[3-(4-aminophenoxy)phenyl]ketone, bis[4-(3-aminophenoxy)phenyl]ketone, bis[4-(4-aminophenoxy)phenyl]ketone, bis[3-(3-aminophenoxy)phenyl]sulfide, bis[3-(4-aminophenoxy)phenyl]sulfide, bis[4-(3-aminophenoxy)phenyl]sulfide, bis[4-(4-aminophenoxy)phenyl]sulfide, bis[3-(3-aminophenoxy)phenyl]sulfone, bis[3-(4-aminophenoxy)phenyl]sulfone, bis[4-(3-aminophenoxy)phenyl]sulfone, bis[4-(4-aminophenoxy)phenyl]sulfone, bis[3-(3-aminophenoxy)phenyl]methane, bis[3-(4-aminophenoxy)phenyl]methane, bis[4-(3-aminophenoxy)phenyl]methane, bis[4-(4-aminophenoxy)phenyl]methane, 2,2-bis[3-(3-aminophenoxy)phenyl]propane, 2,2-bis[3-(4-aminophenoxy)phenyl]propane, 2,2-bis[4-(3-aminophenoxy)phenyl]propane, 2,2-bis[4-(4-aminophenoxy)phenyl]propane, 2,2-bis[3-(3-aminophenoxy)phenyl]-1,1,1,3,3,3-hexafluoropropane, 2,2-bis[3-(4-aminophenoxy)phenyl]-1,1,1,3,3,3-hexafluoropropane, 2,2-bis[4-(3-aminophenoxy)phenyl]-1,1,1,3,3,3-hexafluoropropane and 2,2-bis[4-(4-aminophenoxy)phenyl]-1,1,1,3,3,3-hexafluoropropane.

These may be used alone or in mixtures of two or more. The diamine used may be appropriately selected according to the properties desired.

Preferred among these are aromatic diamine compounds, with 3,3′-diaminodiphenyl ether, 3,4′-diaminodiphenyl ether, 4,4′-diaminodiphenyl ether, paraphenylenediamine, 1,3-bis(3-aminophenyl)benzene, 1,3-bis(4-aminophenyl)benzene, 1,4-bis(3-aminophenyl)benzene, 1,4-bis(4-aminophenyl)benzene, 1,3-bis(4-aminophenoxy)benzene and 1,4-bis(3-aminophenoxy)benzene being preferred for use. Particularly preferred is at least one type of diamine selected from the group consisting of benzenediamines, diaminodiphenyl ethers and bis(aminophenoxy)phenyl.

From the viewpoint of heat resistance and dimensional stability under high temperature, the porous polyimide film which may be used for the invention is preferably formed from a polyimide obtained by combination of a tetracarboxylic dianhydride and a diamine, having a glass transition temperature of 240° C. or higher, or without a distinct transition point at 300° C. or higher.

From the viewpoint of heat resistance and dimensional stability under high temperature, the porous polyimide film which may be used for the invention is preferably a porous polyimide film comprising one of the following aromatic polyimides.

(i) An aromatic polyimide comprising at least one tetracarboxylic acid unit selected from the group consisting of biphenyltetracarboxylic acid units and pyromellitic acid units, and an aromatic diamine unit,

(ii) an aromatic polyimide comprising a tetracarboxylic acid unit and at least one type of aromatic diamine unit selected from the group consisting of benzenediamine units, diaminodiphenyl ether units and bis(aminophenoxy)phenyl units, and/or,

(iii) an aromatic polyimide comprising at least one type of tetracarboxylic acid unit selected from the group consisting of biphenyltetracarboxylic acid units and pyromellitic acid units, and at least one type of aromatic diamine unit selected from the group consisting of benzenediamine units, diaminodiphenyl ether units and bis(aminophenoxy)phenyl units.

The porous polyimide film used in the present invention is preferably a three-layer structure porous polyimide film having a surface layer A and a surface layer B, the surface layers having a plurality of pores, and a macrovoid layer sandwiched between the surface layers A and B; wherein an average pore diameter of the pores present in the surface layer A is 0.01 μm to 15 μm, and the mean pore diameter present on the surface layer B is 20 μm to 100 μm; wherein the macrovoid layer has a partition wall bonded to the surface layers A and B, and a plurality of macrovoids surrounded by such a partition wall and the surface layers A and B; wherein the thickness of the macrovoid layer, and the surface layers A and B is 0.01 to 20 μm, wherein the pores on the surface layers A and B communicate with the macrovoid, the total film thickness is 5 to 500 μm, and the porosity is 40% or more and less than 95%. In this case, at least one partition wall in the macrovoid layer has one or two or more pores connecting the neighboring macrovoids and having the average pore diameter of 0.01 to 100 μm, preferably 0.01 to 50 μm.

For example, porous polyimide films described in WO2010/038873, Japanese Unexamined Patent Publication (Kokai) No. 2011-219585 or Japanese Unexamined Patent Publication (Kokai) No. 2011-219586 may be used for the present invention.

1-2. Porous Polyethersulfone Film (Porous PES Film)

The porous polyethersulfone film which may be used for the present invention contains polyethersulfone and typically consists substantially of polyethersulfone. Polyethersulfone may be synthesized by the method known to those skilled in the art. For example, it may be produced by a method wherein a dihydric phenol, an alkaline metal compound and a dihalogenodiphenyl compound are subjected to polycondensation reaction in an organic polar solvent, a method wherein an alkaline metal di-salt of a dihydric phenol previously synthesized is subjected to polycondensation reaction dihalogenodiphenyl compound in an organic polar solvent or the like.

Examples of an alkaline metal compound include alkaline metal carbonate, alkaline metal hydroxide, alkaline metal hydride, alkaline metal alkoxide and the like. Particularly, sodium carbonate and potassium carbonate are preferred.

Examples of a dihydric phenol compound include hydroquinone, catechol, resorcin, 4,4′-biphenol, bis (hydroxyphenyl)alkanes (such as 2,2-bis(hydroxyphenyl)propane, and 2,2-bis(hydroxyphenyl)methane), dihydroxydiphenylsulfones, dihydroxydiphenyl ethers, or those mentioned above having at least one hydrogen on the benzene rings thereof substituted with a lower alkyl group such as a methyl group, an ethyl group, or a propyl group, or with a lower alkoxy group such as a methoxy group, or an ethoxy group. As the dihydric phenol compound, two or more of the aforementioned compounds may be mixed and used.

Polyethersulfone may be a commercially available product. Examples of a commercially available product include SUMIKAEXCEL 7600P, SUMIKAEXCEL 5900P (both manufactured by Sumitomo Chemical Company, Limited).

The logarithmic viscosity of the polyethersulfone is preferably 0.5 or more, more preferably 0.55 or more from the viewpoint of favorable formation of a macrovoid of the porous polyethersulfone film; and it is preferably 1.0 or less, more preferably 0.9 or less, further preferably 0.8 or less, particularly preferably 0.75 or less from the viewpoint of the easy production of a porous polyethersulfone film.

Further, from the viewpoints of heat resistance and dimensional stability under high temperature, it is preferred that the porous polyethersulfone film or polyethersulfone as a raw material thereof has a glass transition temperature of 200° C. or higher, or that a distinct glass transition temperature is not observed.

The method for producing the porous polyethersulfone film which may be used for the present invention is not particularly limited. For example, the film may be produced by a method including the following steps:

a step in which polyethersulfone solution containing 0.3 to 60% by mass of polyethersulfone having logarithmic viscosity of 0.5 to 1.0 and 40 to 99.7% by mass of an organic polar solvent is casted into a film, immersed in or contacted with a coagulating solvent containing a poor solvent or non-solvent of polyethersulfone to produce a coagulated film having pores; and

a step in which the coagulated film having pores obtained in the above-mentioned step is heat-treated for coarsening of the aforementioned pores to obtain a porous polyethersulfone film;

wherein the heat treatment includes the temperature of the coagulated film having the pores is raised higher than the glass transition temperature of the polyethersulfone, or up to 240° C. or higher.

The porous polyethersulfone film which can be used in the present invention is preferably a porous polyethersulfone film having a surface layer A, a surface layer B, and a macrovoid layer sandwiched between the surface layers A and B,

wherein the macrovoid layer has a partition wall bonded to the surface layers A and B, and a plurality of macrovoids surrounded by such a partition wall and the surface layers A and B, the macrovoids having the average pore diameter in the planar direction of the film of 10 to 500 μm;

wherein the thickness of the macrovoid layer is 0.1 to 50 each of the surface layers A and B has a thickness of 0.1 to 50 μm,

wherein one of the surface layers A and B has a plurality of pores having the average pore diameter of more than 5 μm and 200 μm or less, while the other has a plurality of pores having the average pore diameter of 0.01 μm or more and less than 200 μm,

wherein one of the surface layers A and B has a surface aperture ratio of 15% or more while other has a surface aperture ratio of 10% or more,

wherein the pores of the surface layers A and B communicate with the macrovoids,

wherein the porous polyethersulfone film has total film thickness of 5 to 500 μm and a porosity of 50 to 95%.

As the aforementioned porous polymer film used as a cell culture support in the cell culture device of the present invention has pores with slightly hydrophilic properties, stable liquid retention is achieved within the porous polymer film and a moist environment that is resistant to drying is maintained. Thus, survival and proliferation of cells can be achieved even with very little medium compared to conventional cell culture devices that use a cell culture support. Furthermore, as culturing is possible even if a part of or the entire porous polymer film is exposed to air, oxygen can be efficiently supplied to the cells and a large number of cells can be cultured.

According to the present invention, very little medium is used, and as the porous polymer film used as the culture support can be exposed to a gaseous phase, the supply of oxygen to the cells can be sufficiently performed through diffusion. Thus, there is no particular need for an oxygen supply device in the present invention.

### 2. Cell Culture Device

The present invention relates to a cell culture device comprising: two or more parallel or antiparallel levels of planar flow paths; porous polymer films provided on the planar flow paths; medium supply means each provided at one end of each of the planar flow paths; medium discharge means each provided at the other end of each of the planar flow paths; a head tank provided on a part above the uppermost level of the planar flow paths; a medium distribution means for distributing culture liquid to the planar flow paths from the head tank; and a medium recovery means for recovering culture liquid discharged from the medium discharge means. The porous polymer films provided on the planar flow paths constituting the cell culture device are each a three-layer structured porous polymer film having a surface layer A and a surface layer B having a plurality of pores, and a macrovoid layer provided between the surface layer A and the surface layer B, the average pore diameter of the pores present in the surface layer A is smaller than the average pore diameter of the pores present in the surface layer B, and the macrovoid layer has a partition wall that is bound to the surface layers A and B, and has a plurality of macrovoids enclosed by the partition wall and the surface layers A and B. The cell culture device according to the present invention includes embodiments which are a parallel-type cell culture device and a cascade-type cell culture device depending on the arrangement of the planar flow paths. In general, these cell culture devices are referred to herein as the “cell culture device of the present invention”. Embodiments of the cell culture device of the present invention will be described below with reference to the drawings.

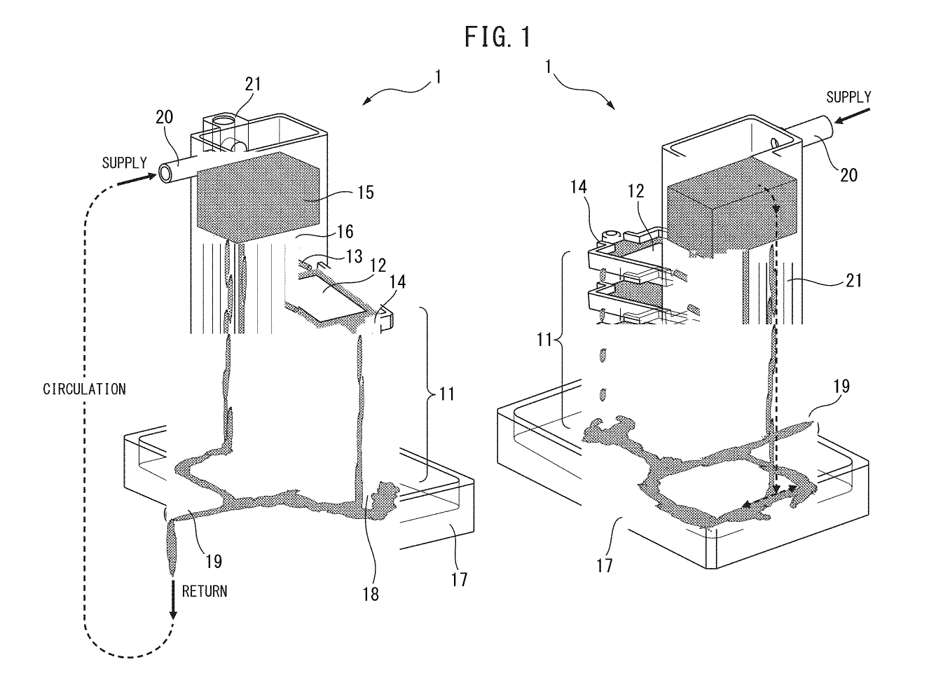

FIG. 1 is a drawing illustrating a typical example of a parallel-type cell culture device according to the present invention. Planar flow paths 11 are provided with porous polymer films 12 disposed thereon and medium discharge means. In one embodiment, medium supply means 13 may be provided on the planar flow paths 11 or may be connected to medium distribution means 16. The planar flow paths 11 may be provided horizontally or inclined and are preferably between 0 and 20° from the horizontal plane, for example, in the ranges of 0 to 15°, 5 to 20°, 5 to 15°, and 10 to 15°, or any of 0°, 1°, 2°, 3°, 4°, 5°, 6°, 7°, 8°, 9°, 10°, 11°, 12°, 13°, 14°, 15°, 16°, 17°, 18°, 19°, and 20°. The number of levels of the planar flow paths 11 used in the cell culture device herein is not limited provided that the cell culture device is configured from two levels or more, and is preferably 2 to 300 levels, more preferably 2 to 200 levels, even more preferably 2 to 100 levels. For example, there may be 2 to 90 levels, 80 levels, 70 levels, 60 levels, 50 levels, 40 levels, 30 levels, 20 levels, 10 levels, 5 levels, 4 levels, and 3 levels or less. Furthermore, the positions and sizes of the planar flow paths 11 may be changed appropriately according to the purpose. Note that the whole of each of the planar flow paths may be provided within a thin layered bag.

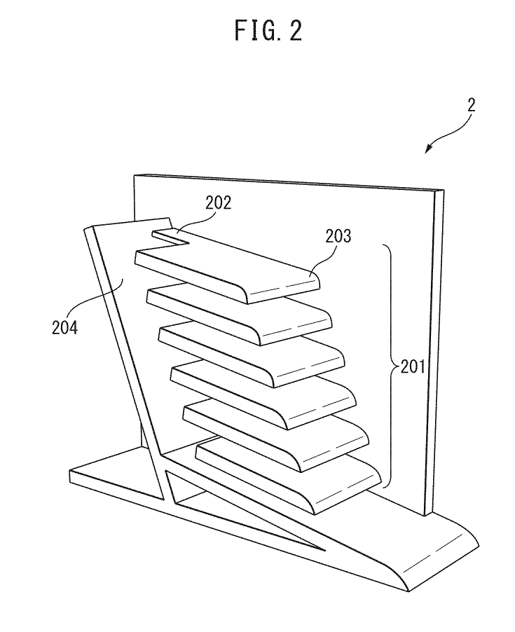

The culture liquid stored in the head tank 15 is supplied over the entirety of the planar flow paths 11 from the medium supply means 13 via the medium distribution means 16. The medium distribution means 16 may each be a tube connected to the head tank 15 or may be an inclined plate 204 as illustrated in FIG. 2. Further, a regulator (not shown) for regulating the flow of culture liquid fed to the entirety of the planar flow paths through the medium supply means 13 from the head tank 15 via the medium distribution means 16 may be provided on the head tank 15, each medium distribution means 16, or on each medium supply means 13. The culture liquid supplied from the medium supply means soaks the porous polymer film provided on the planar flow paths 11 and flows toward the medium discharge means 14. Note that the medium supply means 13, which are not shown, may each be a mist supply nozzle that can supply the culture liquid as fine droplets. In the present invention, provided the mist supply nozzle is a means that supplies a medium as droplets, there is no limit to the number of nozzles, the arrangement thereof, or the size of the droplets supplied thereby.

The culture liquid discharged from the medium discharge means 14 may be directly dripped onto the medium recovery means 17 for recovering culture liquid in order to recover the medium or may be recovered in the medium recovery means 17 along a drip rod 18 (surplus liquid return rod) provided on a bottom part of the medium recovery means 17 that makes contact with each of the medium discharge means 14.

In one embodiment, the cell culture device may further comprise a medium discharge line in communication with a discharge port 19 of the medium recovery means 17, and a medium supply line in communication with a supply port 20 provided on an upper part of the head tank 15. The other end of the medium discharge line is in communication with the other end of the medium supply line via a pump and the medium can be circulated. The type of pump for pumping up the culture liquid is not particularly limited but, for example, a peristaltic pump with which the flow rate can be regulated can be used. Furthermore, in order to prevent leaking of culture liquid supplied from the medium supply line from the head tank 15, the head tank 15 may be provided with an overflow line 21.

FIG. 2 is a conceptual diagram illustrating an embodiment of the parallel-type cell culture device wherein the planar plates 201, medium supply plates 202, medium discharge plates 203, and inclined plate 204 correspond to the planar flow paths, the medium supply means 13, the medium discharge means 14, and the medium distribution means 16 of FIG. 1.

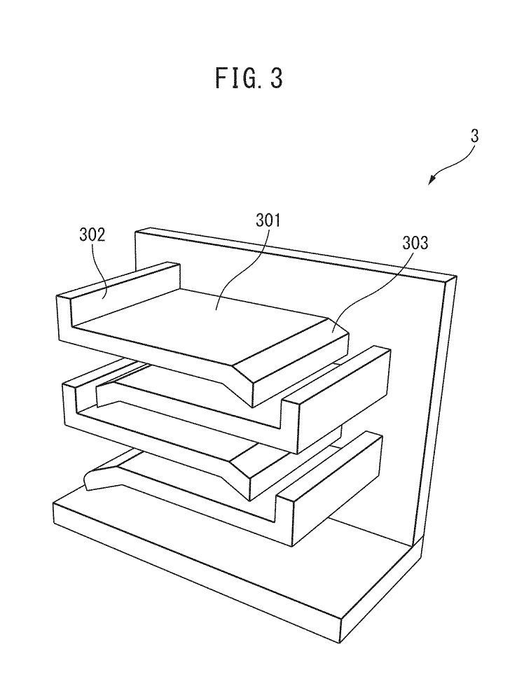

FIG. 3 is a conceptual diagram of a cascade-type cell culture device of the present invention. The cascade-type cell culture device is characterized in that culture liquid discharged from the medium discharge plate 303 provided on one planar plate 301 pours onto the medium supply plate 302 provided on the planar plate 301 on the next level. A person skilled in the art could confirm that the porous polymer film, planar flow path, head tank, medium recovery means, each type of line, and pump that constitute the parallel-type cell culture device can be appropriately used for the cascade-type cell culture device.

The porous polymer film used in an embodiment of the present invention may, for example, be: i) folded up; ii) wound into a roll shape; iii) connected to a sheet or small piece by a filamentous structure; and/or iv) tied in a rope shape; and is provided in the planar flow path. Further, the porous polymer film used in the present embodiment may be v) laminated in two or more layers and provided in the planar flow path. By processing the porous polymer films into forms like i) to v), many porous polymer films can be added to a fixed quantity of cell culture medium.

The porous polymer film used in an embodiment of the present invention, may be a modularized porous polymer film (referred to as “modularized porous polymer film” below). The “modularized porous polymer film” herein is a porous polymer film accommodated in a casing.

The casing provided with a modularized porous polymer film used in an embodiment of the present invention has two or more cell medium flow ports, and the medium flows into or out of the casing through the cell culture medium flow ports. The diameter of the medium flow inlet of the casing is preferably larger than the diameter of the cell so as to enable cell to flow into the casing. In addition, the diameter of the medium flow inlet is preferably smaller than the diameter through which the porous polymer film flows out from the medium flow inlet. The diameter smaller than the diameter through which the porous polymer film flows out may be appropriately selected depending on the shape and size of the porous polymer film contained in the casing. For example, when the porous polymer film has string-like shape, the diameter is not particularly limited so long as it is smaller than the width of the shorter side of the porous polymer film so that the porous polymer film is prevented from flowing out. It is preferred to provide as many medium flow inlets as possible so that the cell culture medium may be easily supplied into and/or discharged from the casing. It is preferably 5 or more, preferably 10 or more, preferably 20 or more, preferably 50 or more, and preferably 100 or more. As for the medium flow inlet, the casing may have a mesh-like structure in part or as a whole. Moreover, the casing itself may be mesh-like. In the present invention, examples of mesh-like structure include, but not limited to, those including longitudinal, transverse, and/or oblique elements wherein individual apertures form medium flow inlets which allow the fluid to pass therethrough.

The casing for the modularized porous polymer film used in an embodiment of the present invention may be, for example, polystyrene, polycarbonate, polymethylmethacrylate, polyethyleneterephthalate, and provided the culturing of cells is not affected, there are no particular restrictions on the material.

The modularized porous polymer film used in an embodiment of the present invention is accommodated in the casing such that:

i) two or more individual porous polymer films are aggregated;

ii) the porous polymer film is folded;

iii) the porous polymer film is wound into a roll shape; and/or

iv) the porous polymer film is tied into a rope shape; and

wherein the modularized porous polymer film may be provided in the planar flow path.

Herein, “two or more individual porous polymer films are aggregated and accommodated within a casing” describes a state in which two or more porous polymer films that are separate from each other are aggregated and accommodated within a fixed space enclosed by the casing. In the present invention two or more individual porous polymer films may be secured so that the porous polymer films do not move in the casing by securing at least one part of the porous polymer films to at least one part of the casing interior using an arbitrary method. Furthermore, the two or more individual porous polymer films may be small pieces. The shape of the small pieces may be any shape such as a circle, ellipse, quadrilateral, triangle, polygonal or string-like but is preferably a square. In the present invention, the size of the small piece may be any size, but in the case of a square, the length may be any length but the width is preferably 80 mm or less, preferably 30 mm or less, and more preferably 10 mm or less. This prevents stress from being applied to cells grown in the porous polymer film.

In this specification, “the porous polymer films being folded up” means a porous polymer film which is folded up in the casing, and thus it is rendered immovable in the casing by frictional force between each surfaces of the porous polymer film and/or the inner surface of the casing. In this specification, “being folded” may indicate the pours polymer film being creased or creaseless.

In this specification, “the porous polymer films being wound into a roll-like shape” means the porous polymer film being wound into a roll-like shape and thus it is rendered immovable in the casing by frictional force between each surfaces of the porous polymer film and/or the inner surface of the casing. Moreover, in the present invention, the porous polymer film being tied together into a rope-like shape means, for example, more than one porous polymer films in rectangle strip shape are knitted into a rope-shape by arbitrary method, rendering the porous polymer films immovable by the mutual frictional force of the porous polymer films. It is also possible that (i) the two or more independent porous polymer films being aggregated; (ii) the porous polymer films being folded up; (iii) the porous polymer films being wound into a roll-like shape; and/or (iv) the porous polymer film being tied together into a rope-like shape may be combined and contained within a casing.

In this specification, “the porous polymer film being immovable in the casing” means that the porous polymer film is contained in the casing so that the porous polymer film is continually morphologically unchanged during culturing the cell culture module in the cell culture medium. In other words, the porous polymer film itself is continually prevented from waving by fluid. Since the porous polymer film is kept immovable in the casing, the cell growing in the porous polymer film is protected from stress to be applied, enabling stable cell culture without cells being killed by apoptosis.

### 3. Cell Culture Method Using Cell Culture Device

<Step of Applying Cells to Porous Polymer Film>

The specific step by which the cells used in the present invention are applied to the porous polymer film is not particularly limited. The process described herein or any suitable means for applying cells to a film-like support may be adopted. The means for applying the cells to the porous polymer film in the method of the present invention include the following embodiments but are not limited thereby.

(A) An embodiment including the step of inoculating cells on the surface of a porous polymer film.

(B) An embodiment including the step of placing a cell suspension onto the surface of a dried porous polymer film which is either left standing, moved to promote the outflow of liquid, or a part of the surface thereof is stimulated so that the cell suspension is absorbed into the film such that the cells in the cell suspension are retained in the film and the moisture flows out.

(C) An embodiment including the step of moistening one or both surfaces of the porous polymer film with the cell culture liquid or a sterilized liquid and loading the moistened porous polymer film with the cell suspension such that the cells in the cell suspension are retained in the film and the moisture flows out.

Embodiment (A) includes the direct inoculation of cells or a cell cluster onto the surface of the porous polymer film. Alternatively, an embodiment in which the porous polymer film is placed in the cell suspension such that the cell culture liquid permeates the film surface is also included.

The cells inoculated onto the surface of the porous polymer film adhere to the porous polymer film and permeate into the pores. The cells preferably adhere to the porous polymer film without the addition of any particular external physical or chemical forces. The cells inoculated onto the surface of the porous polymer film can stably grow and proliferate in and/or on the surface of the film. A variety of different forms can be achieved according to the position of the film on which the cells grow and proliferate.

In embodiment (B) a cell suspension is placed on the surface of a dried porous polymer film. In order for the cell suspension to permeate into the film, the porous polymer film is left standing, moved to promote the outflow of liquid, or a part of the surface thereof is stimulated so that the cell suspension is absorbed into the film. Although not bound by theory, it is considered that this permeation is dependent on a property derived from the shape, etc., of each surface of the porous polymer film. According to the present embodiment, cells are absorbed into and inoculated in the part of the film loaded with the cell suspension.

Alternatively, like embodiment (C), a part or the whole of one or both surfaces of the porous polymer film may be moistened with cell culture liquid or a sterilized liquid then the cell suspension loaded onto the moistened porous polymer film. In such cases, the permeation rate of the cell suspension substantially increases.

For example, a method of moistening an extremely small part of the film (hereinafter referred to as “one-point wetting method”) focusing on the prevention of splashing from the film can be used. The one-point wetting method is practically very similar to the dry method (embodiment (B)) in which the film is not moistened. However, regarding the small portion that is moistened, it is considered that the passage of the cell solution through the film becomes rapid. Furthermore, a method in which a cell suspension is loaded onto a porous polymer film that is sufficiently moistened over the entirety of one or both surfaces (hereinafter referred to as “wet film”) can also be used (hereinafter referred to as “wet film method”). In such cases, the permeation rate of the cell suspension through the entire porous polymer film substantially increases.

In embodiments (B) and (C), cells in the cell suspension are retained in the film and moisture flows out. The concentration of cells in the cell suspension is thereby increased, and treatment involving, for example, the outflow of unnecessary components that do not include the cells together with the moisture is also possible.

There are cases when embodiment (A) is referred to as “natural inoculation” and embodiments (B) and (C) are referred to as “absorption inoculation”.

It is preferable for living cells to be selectively retained in the porous polymer film although this is in no ways limiting. Thus, in a method of a preferable embodiment of the present invention, living cells are retained in the porous polymer film, and dead cells preferentially flow out with the moisture.

The sterilized liquid used in embodiment (C) is not particularly limited but is a sterilized buffer solution or sterilized water. The buffer solution is, for example, (+) and (−) Dulbecco's PBS, (+) and (−) Hanks's Balanced Salt Solution. Examples of the buffer solution are indicated in Table 1 below.

TABLE 1

| Component | Concentration (mmol/L) | Concentration (g/L) |
|---|---|---|
| NaCl | 137 | 8.00 |
| KCl | 2.7 | 0.20 |
| Na₂HPO₄ | 10 | 1.44 |
| KH₂PO₄ | 1.76 | 0.24 |
| pH(−) | 7.4 | 7.4 |

Furthermore, the method according to the present invention includes an embodiment (entwining) wherein the application of the cells to the porous polymer film involves adhering the cells onto the film by making suspended adhesive cells coexist with the porous polymer film in suspension. For example, in a method according to the present invention, in order to apply the cells to the porous polymer film, a cell culture medium, cells and one or more porous polymer films may be added to a cell culture vessel. When the cell culture medium is liquid, the porous polymer film is suspended in the cell culture medium. Due to the properties of the porous polymer film the cells are able to adhere to the porous polymer film. Thus, even if the cells are not suited to natural suspension cultures, culturing is possible with the porous polymer film suspended in the cell culture medium. The cells are preferably adhered to the porous polymer film. “Spontaneously adhere” is defined as the retention of cells on the surface or in the interior of the porous polymer film without the addition of any particular external physical or chemical forces.

The aforementioned application of cells to the porous polymer film may include the combination of two or more methods. For example, the cells may be applied to the porous polymer film by combining two or more methods from among embodiments (A) to (C). It is possible to culture cells supported on the porous polymer film by the application thereof to the planar flow paths of the aforementioned cell culture device.

In addition, a medium in which cells are suspended may be inoculated as droplets by a cell supply means onto the planar flow path on which is provided the porous polymer film in advance.

“Suspended cells” as defined herein include adherent cells that can be suspension cultured in a medium wherein the cells are obtained by, for example, forcibly suspending adherent cells in a medium by using a proteolytic enzyme such as trypsin or by using a publicly-known conditioning step.

The types of the cells which may be used for the present invention may be selected from the group consisting of animal cells, insect cells, plant cells, yeast cells and bacteria. Animal cells are largely divided into cells from animals belonging to the subphylum Vertebrata, and cells from non-vertebrates (animals other than animals belonging to the subphylum Vertebrata). There are no particular restrictions on the source of the animal cells, for the purpose of the present specification. Preferably, they are cells from an animal belonging to the subphylum Vertebrata. The subphylum Vertebrata includes the superclass Agnatha and the superclass Gnathostomata, the superclass Gnathostomata including the class Mammalia, the class Ayes, the class Amphibia and the class Reptilia. Preferably, they are cells from an animal belonging to the class Mammalia, generally known as mammals. Mammals are not particularly restricted but include, preferably, mice, rats, humans, monkeys, pigs, dogs, sheep and goats.

The types of animal cells or plant cells that may be used for the invention are not particularly restricted, but are preferably selected from the group consisting of pluripotent stem cells, tissue stem cells, somatic cells and germ cells.

The term “pluripotent stem cells”, in this specification, is intended as a comprehensive term for stem cells having the ability to differentiate into cells of any tissues (pluripotent differentiating power). While not restrictive, pluripotent stem cells include embryonic stem cells (ES cells), induced pluripotent stem cells (iPS cells), embryonic germ cells (EG cells) and germ stem cells (GS cells). They are preferably ES cells or iPS cells. Particularly preferred are iPS cells, which are free of ethical problems, for example. The pluripotent stem cells used may be any publicly known ones, and for example, the pluripotent stem cells described in WO2009/123349 (PCT/JP2009/057041) may be used.

The term “tissue stem cells” refers to stem cells that are cell lines capable of differentiation but only to limited specific tissues, though having the ability to differentiate into a variety of cell types (pluripotent differentiating power). For example, hematopoietic stem cells in the bone marrow are the source of blood cells, while neural stem cells differentiate into neurons. Additional types include hepatic stem cells from which the liver is formed and skin stem cells that form skin tissue. Preferably, the tissue stem cells are selected from among mesenchymal stem cells, hepatic stem cells, pancreatic stem cells, neural stem cells, skin stem cells and hematopoietic stem cells.

The term “somatic cells” refers to cells other than germ cells, among the cells composing a multicellular organism. In sexual reproduction, these are not passed on to the next generation. Preferably, the somatic cells are selected from among hepatocytes, pancreatic cells, muscle cells, bone cells, osteoblasts, osteoclasts, chondrocytes, adipocytes, skin cells, fibroblasts, pancreatic cells, renal cells and lung cells, or blood cells such as lymphocytes, erythrocytes, leukocytes, monocytes, macrophages or megakaryocytes.

The term “germ cells” refers to cells having the role of passing on genetic information to the succeeding generation in reproduction. These include, for example, gametes for sexual reproduction, i.e. the ova, egg cells, sperm, sperm cells, and spores for asexual reproduction.

The cells may also be selected from the group consisting of sarcoma cells, established cell lines and transformants. The term “sarcoma” refers to cancer occurring in non-epithelial cell-derived connective tissue cells, such as the bone, cartilage, fat, muscle or blood, and includes soft tissue sarcomas, malignant bone tumors and the like. Sarcoma cells are cells derived from sarcoma. The term “established cell line” refers to cultured cells that are maintained in vitro for long periods and reach a stabilized character and can be semi-permanently subcultured. Cell lines derived from various tissues of various species including humans exist, such as PC12 cells (from rat adrenal medulla), CHO cells (from Chinese hamster ovary), HEK293 cells (from human embryonic kidney), HL-60 cells (from human leukocytes) and HeLa cells (from human cervical cancer), Vero cells (from African green monkey kidney epithelial cells), MDCK cells (from canine renal tubular epithelial cells), HepG2 cells (from human hepatic cancer), BHK cells (new-born hamster kidney cell), NIH3T3 cells (from mouse fetal fibroblast cells). The term “transformants” refers to cells with an altered genetic nature by extracellularly introduced nucleic acid (DNA and the like).

In this specification, an “adherent cell” is generally a cell which is required to adhere itself on an appropriate surface for growth, and is also referred to as an adhesion cell or an anchorage-dependent cell. In certain embodiments of the present invention, the cells used are adherent cells. The cells used for the present invention are adherent cells, more preferably cells which may be cultured even as a suspension in a medium. The adherent cells which can be suspension cultured may be obtained by conditioning the adherent cells to a state suitable for suspension culture, and include, for example, CHO cells, HEK293 cells, Vero cells, NIH3T3 cells, and cell lines derived from these cells. The cells used for the present invention other than listed herein are not particularly limited so long that they may be applied to suspension culture by conditioning.

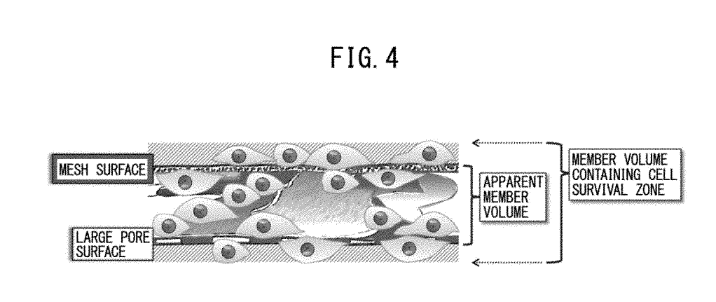

FIG. 4 represents a model diagram of cell culturing using a porous polymer film. FIG. 4 serves merely for illustration and the elements are not drawn to their actual dimensions. In the cell culture method of the invention, application of cells and culturing are carried out on a porous polymer film, thereby allowing culturing of large volumes of cells to be accomplished since large numbers of cells grow on the multisided connected pore sections on the inside, and the surfaces on the porous polymer film. Moreover, in the cell culture method of the invention, it is possible to culture large volumes of cells while drastically reducing the amount of medium used for cell culturing compared to the prior art. For example, large volumes of cells can be cultured even when all or a portion of the porous polymer film is not in contact with the liquid phase of the cell culture medium. In addition, the total volume of the cell culture medium in the cell culture vessel, with respect to the total porous polymer film volume including the cell survival zone, can be significantly reduced.

Throughout the present specification, the volume of the porous polymer film without cells, that occupies the space including the volume between the interior gaps, will be referred to as the “apparent porous polymer film volume” (see, FIG. 4). In the state where the cells are applied to the porous polymer film and the cells have been supported on the surface and the interior of the porous polymer film, the total volume of the porous polymer film, the cells and the medium that has wetted the porous polymer film interior, which is occupying the space therein, will be referred to as the “porous polymer film volume including the cell survival zone” (see, FIG. 1). When the porous polymer film has a film thickness of 25 μm, the porous polymer film volume including the cell survival zone is a value of at maximum about 50% larger than the apparent porous polymer film volume. In the method of the invention, a plurality of porous polymer films may be housed in a single cell culture vessel for culturing, in which case the total sum of the porous polymer film volume including the cell survival zone for each of the plurality of porous polymer films supporting the cells may be referred to simply as the “total sum of the porous polymer film volume including the cell survival zone”.

Using the method of the invention, cells can be satisfactorily cultured for a long period of time even under conditions in which the total volume of the cell culture medium in the cell culture vessel is 10,000 times or less of the total sum of the porous polymer film volume including the cell survival zone. Moreover, cells can be satisfactorily cultured for a long period of time even under conditions in which the total volume of the cell culture medium in the cell culture vessel is 1,000 times or less of the total sum of the porous polymer film volume including the cell survival zone. In addition, cells can be satisfactorily cultured for a long period of time even under conditions in which the total volume of the cell culture medium in the cell culture vessel is 100 times or less of the total sum of the porous polymer film volume including the cell survival zone. In addition, cells can be satisfactorily cultured for a long period of time even under conditions in which the total volume of the cell culture medium in the cell culture vessel is 10 times or less of the total sum of the porous polymer film volume including the cell survival zone.

In other words, according to the invention, the space (vessel) used for cell culturing can be reduced to an absolute minimum, compared to a conventional cell culture device for performing two-dimensional culture. Furthermore, when it is desired to increase the number of cells cultured, the cell culturing volume can be flexibly increased by a convenient procedure including increasing the number of layered porous polymer films. In a cell culture device comprising a porous polymer film to be used for the invention, the space (vessel) in which cells are cultured and the space (vessel) in which the cell culture medium is stored can be separate, and the necessary amount of cell culture medium can be prepared according to the number of cells to be cultured. The space (vessel) in which the cell culture medium is stored can be increased or decreased according to the purpose, or it may be a replaceable vessel, with no particular restrictions.

In the cell culture method of the invention, culturing in which the number of cells in the cell culture vessel after culturing using the porous polymer film reaches 1.0×10⁵ or more, 1.0×10⁶ or more, 2.0×10⁶ or more, 5.0×10⁶ or more, 1.0×10⁷ or more, 2.0×10⁷ or more, 5.0×10⁷ or more, 1.0×10⁸ or more, 2.0×10⁸ or more, 5.0×10⁸ or more, 1.0×10⁹ or more, 2.0×10⁹ or more, or 5.0×10⁹ or more per milliliter of medium, assuming that all of the cells are evenly dispersed in the cell culture medium in the cell culture vessel, is mentioned.

It should be noted that as a method for measuring cell count during or after culture, various known methods may be used. For example, as the method for counting the number of cells in the cell culture vessel after culturing using the porous polymer film, assuming that the cells are evenly dispersed in the cell culture medium in the cell culture vessel, any publicly known method may be used. For example, a cell count method using CCK8 may be suitably used. Specifically, a Cell Counting Kit 8 (a solution reagent, commercially available from Dojindo Laboratories)(hereunder referred to as “CCK8”) may be used to count the number of cells in ordinary culturing without using a porous polymer film, and the correlation coefficient between the absorbance and the actual cell count is determined. Subsequently, the cells are applied, the cultured porous polymer film may be transferred to CCK8-containing medium and stored in an incubator for 1 to 3 hours, and then the supernatant is extracted and its absorbance is measured at a wavelength of 480 nm, and the cell count is determined from the previously calculated correlation coefficient.

In addition, from another point of view, for example, “mass culturing of cells” may refer to culturing in which the number of cells in the cell culture vessel after culturing using the porous polyimide film reaches 1.0×10⁵ or more, 2.0×10⁵ or more, 1.0×10⁶ or more, 2.0×10⁶ or more, 5.0×10⁶ or more, 1.0×10⁷ or more, 2.0×10⁷ or more or 5.0×10⁷ or more, 1.0×10⁸ or more, 2.0×10⁸ or more, or 5.0×10⁸ or more, per square centimeter of porous polymer film. The number of cells contained per square centimeter of porous polymer film may be appropriately measured using a publicly known method, such as with a cell counter.

### EXAMPLES

The present invention will now be explained in greater detail by Examples. It is to be understood, however, that the invention is not limited to these Examples. A person skilled in the art may easily implement modifications and changes to the invention based on the description in the present specification, and these are also encompassed within the technical scope of the invention.

The porous polyimide films used in the following examples were prepared by forming a polyamic acid solution composition including a polyamic acid solution obtained from 3,3′,4,4′-biphenyltetracarboxylic dianhydride (s-BPDA) as a tetracarboxylic acid component and 4,4′-diaminodiphenyl ether (ODA) as a diamine component, and polyacrylamide as a coloring precursor, and performing heat treatment at 250° C. or higher. The resulting porous polyimide film was a three-layer structure porous polyimide film having a surface layer A and a surface layer B, the surface layers having a plurality of pores, and a macrovoid layer sandwiched between the surface layers A and B; wherein the average pore diameter of the pore present on the surface layer A was 6 μm, the average pore diameter of the pore present on the surface layer B was 46 μm, and the film thickness was 25 μm, and the porosity was 73%.

### Example 1

Cell Culture Method Using Porous Polyamide Films in Parallel Flow Reactor (FIG. 1)

Conditioned and suspended anti-human IL-8 antibody producing CHO-DP12 cells (ATCC CRL-12445) were suspension cultured using a medium (BalanCD (trademark) CHO Growth A) until the number of viable cells per ml reached 1.5×10⁶. 12 ml of the suspension culture liquid was added to each of five 10 cm diameter petri dishes, thereafter, six 2.8 cm×2 cm rectangular porous polyamide films were immersed in each petri dish and left standing in an incubator for four hours. The cell density (cells/ml) in each petri dish after four hours was 3.1×10⁵, 1.5×10⁵, 1.6×10⁵, 1.4×10⁵, and 2.2×10⁵, each was stable and the number of cells was approximately 90% less than the suspension. The six films from each petri dish were placed in each level of the parallel flow reactor illustrated in FIG. 1, and 150 ml of a medium (I MDM comprising FBS 2%) was stored in the liquid storage part, and the same medium was circulated at a rate of 10 ml per minute through a tube pump. The medium was exchanged after five days and cell culturing was terminated on the eighth day. The cell number measurement results are shown in Table 2. A highly dense cell culture was brought about highly efficiently with a simple and compact cell culture device that does not use an oxygen supply device.

TABLE 2

| | | Total number of cells |
|---|---|---|
| Number of days | Cell density (cell/cm²) | (total for five levels) |
| Fifth day | 4.5 × 10⁵ | 7.5 × 10⁷ |
| Eighth day | 6.3 × 10⁵ | 1.1 × 10⁸ |

### Example 2

Cell Culture Method with Porous Polyamide Films Using Cascade Reactor (FIG. 3)

Conditioned and suspended anti-human IL-8 antibody producing CHO-DP12 cells (ATCC CRL-12445) were suspension cultured using a medium (BalanCD (trademark) CHO Growth A) until the number of viable cells per ml reached 6.0×10⁵. 12 ml of the suspension culture liquid was added to a 10 cm diameter petri dish, thereafter, twenty 1.8 cm×2 cm rectangular porous polyamide films were immersed in the petri dish and left standing in an incubator overnight. The cell density (cells/ml) in the petri dish the following day was 2.1×10⁵ and the number of cells was approximately 65% less. In the cascade-type compact reactor shown in FIG. 3, sheets were placed on the stage so that there would be five for each level, and 150 ml of a medium (I MDM comprising FBS 2%) was stored in the liquid storage part, and the same medium was circulated at a rate of 10 ml per minute through a tube pump. The medium was exchanged after six days and 10 ml of feed medium (BalanCD Feed 1) and 1 ml of a 0.5 N sodium hydroxide solution were added. Cell culturing was terminated on the twelfth day and cell number measurements were carried out using CCK 8. The total cell number was 1.3×10⁸, and the cell density per square centimeter was 1.8×10⁶.

### REFERENCE SIGNS LIST

1 parallel-type cell culture device (first)
11 planar flow paths
12 porous polymer film
13 medium supply means
14 medium discharge means
15 head tank
16 medium distribution means
17 medium recovery means
18 drip rod
19 discharge port
20 supply port
21 overflow line
2 parallel-type cell culture device (second)
201 planar plate
202 medium supply plate
203 medium discharge plate
204 inclined plate
3 cascade-type cell culture device
301 planar plate
302 medium supply plate
303 medium discharge plate

1. A cell culture device characterized by comprising:
two or more-step parallel planar flow paths;
a porous polymer film disposed in the planar flow paths;
a medium supplying means disposed at one end of each of the planar flow paths;
a medium discharging means disposed at the other end of each of the planar flow paths;
a head tank disposed at the top of the uppermost planar flow path;
a medium distributing means for distributing the culture liquid from the head tank to each planar flow path; and
a medium collecting means for collecting the culture liquid discharged from the medium discharging means;
wherein the porous polymer films are a three-layer structure porous polymer film having a surface layer A and a surface layer B, the surface layers having a plurality of pores, and a macrovoid layer sandwiched between the surface layers A and B;
wherein an average pore diameter of the pores present in the surface layer A is smaller than an average pore diameter of the pores present in the surface layer B;
wherein the macrovoid layer has a partition wall bonded to the surface layers A and B, and a plurality of macrovoids surrounded by such a partition wall and the surface layers A and B; and
wherein the culture medium continuously flows from the medium supplying means to the medium discharging means via the planar flow path.

2. A cell culture device characterized by comprising:
two or more-step antiparallel planar flow paths;
a porous polymer film disposed in the planar flow paths;
a medium supplying means disposed at one end of each of the planar flow paths;
a medium discharging means disposed at the other end of each of the planar flow paths;
a head tank disposed at the top of the uppermost planar flow path;
a medium distributing means for distributing the culture liquid from the head tank to each of uppermost planar flow path; and
a medium collecting means for collecting the culture liquid discharged from the lowermost;
wherein the porous polymer film is a three-layer structure porous polymer film having a surface layer A and a surface layer B, the surface layers having a plurality of pores, and a macrovoid layer sandwiched between the surface layers A and B;
wherein an average pore diameter of the pores present in the surface layer A is smaller than an average pore diameter of the pores present in the surface layer B;
wherein the macrovoid layer has a partition wall bonded to the surface layers A and B, and a plurality of macrovoids surrounded by such a partition wall and the surface layers A and B; and
wherein the culture liquid continuously flows from the medium supplying means to the medium discharging means via the planar flow path, and furthermore the culture liquid discharged from the medium discharging means is poured into the medium supplying means installed in the next stage planar flow path.

3. The cell culture device according to claim 1 or 2, wherein the planar flow path is inclined at 0 to 20° from a horizontal plane.

4. The cell culture device according to any one of claims 1 to 3, the device further comprising:
a medium discharging line communicating with an outlet of the medium collecting means; and
a medium supplying line communicating with a supply port disposed at an upper portion of the head tank;
wherein the other end of the medium discharging line is communicated with the other end of the medium supplying line via a pump, and the culture liquid is circulatable.

5. The cell culture device according to any one of claims 1 to 4, wherein the medium distributing means is a mist supplying nozzle.

6. The cell culture device according to any one of claims 1 to 4, wherein the planar flow paths are each installed within the thin bag.

7. The cell culture device according to any one of claims 1 to 6, wherein the porous polymer films are installed in the plane flow path, with the films:
i) being folded up;
ii) being wound into a roll-like shape;
iii) concatenating sheets or pieces thereof with a thread-like structure;
iv) being tied together into a rope-like shape; and/or
v) two or more thereof being stacked.

8. The cell culture device according to any one of claims 1 to 7, wherein the porous polymer films are modularized porous polymer films fitted with a casing;
wherein the modularized porous polymer films fitted with a casing are contained within the casing with:
the two or more independent porous polymer films being aggregated;
(ii) the porous polymer films being folded up;
(iii) the porous polymer films being wound into a roll-like shape; and/or
(iv) the porous polymer films being tied together into a rope-like shape;
wherein the modularized porous polymer films fitted with the casing are installed in the planar flow path.

9. The cell culture device according to any one of claims 1 to 8, wherein the porous polymer film has a plurality of pores having an average pore diameter of 0.01 to 100 μm.

10. The cell culture device according to any one of claims 1 to 9, wherein an average pore diameter of the surface layer A is 0.01 to 50 μm.

11. The cell culture device according to any one of claims 1 to 10, wherein an average pore diameter of the surface layer B is 20 to 100 μm.

12. The cell culture device according to any one of claims 1 to 11, wherein a total film thickness of the porous polymer film is 5 to 500 μm.

13. The cell culture device according to any one of claims 1 to 12, wherein the porous polymer film is a porous polyimide film.

14. The method according to claim 13, wherein the porous polyimide film is a porous polyimide film comprising a polyimide derived from tetracarboxylic dianhydride and diamine.

15. The cell culture device according to claim 13 or 14, wherein the porous polyimide film is a colored porous polyimide film that is obtained by molding a polyamic acid solution composition comprising a polyamic acid solution derived from tetracarboxylic dianhydride and diamine, and a coloring precursor, and subsequently heat-treating the resultant composition at 250° C. or higher.

16. The cell culture device according to any one of claims 1 to 12, wherein the porous polymer film is a porous polyethersulfone film.

17. A method for culturing a cell which uses the cell culture device according to any one of claims 1 to 16.
